(19) United States
(12) Patent Application Publication
(10) Pub. No.: US 2007/0191449 A1
(43) Pub. Date: Aug. 16, 2007

(54) Use of Benzo-Heteroaryl Sulfamide Derivatives for the Treatment of Depression

(76) Inventor: Virginia L. Smith-Swintosky, Hatfield, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/673,705

(22) Filed: Feb. 12, 2007

Related U.S. Application Data
(60) Provisional application No. 60/773,810, filed on Feb. 15, 2006.

(51) Int. Cl. A61K 31/4184; A61K 31/405; A61K 31/381; A61K 31/343; A61K 31/16

(52) U.S. Cl. 514/397; 514/419; 514/443; 514/469; 514/600

(57) ABSTRACT

The present invention is a method for the treatment of depression comprising administering to a subject in need thereof a therapeutically effective amount of one or more novel benzo-heteroaryl sulfamide derivatives of formula (I) as herein defined. The present invention is directed to a method for the treatment of depression, which includes mono-therapy and alternatively, co-therapy with at least one antidepressant.

### CROSS REFERENCE TO RELATED APPLICATIONS

This application claims the benefit of U.S. Provisional Application 60/773,810, filed on Feb. 15, 2006, which is incorporated by reference herein in it's entirety.

### FIELD OF THE INVENTION

The present invention is directed to the use of benzo-heteroaryl sulfamide derivatives for the treatment of depression, including both mono-therapy and co-therapy with at least one anti-depressant.

### BACKGROUND OF THE INVENTION

Mood disorders are divided into depressive disorders (i.e. unipolar depression) and bipolar disorders. Depressive disorders are further divided into major depressive disorder, dysthymic disorder (or dysthymia) and depressive disorder not otherwise specified (Diagnostic and Statistical Manual of Mental Disorders, 4^{th} Edition, American Psychiatric Association, 1994). Major depressive disorder (or unipolar dpression) is characterized as one or more major depressive episodes, which are defined as depressed mood on a daily basis for a minimum duration of two weeks. An episode may be characterized by sadness, indifference or apathy, or irritability and is usually associated with a change in a number of neurovegetative functions, including sleep patterns, appetite and body weight, motor agitation or retardation, fatigue, impairment in concentration and decision making, feelings of shame or guilt, and thoughts of death or dying (Harrison's Principles of Internal Medicine, 2000). The criteria for a major depressive episode includes five or more symptoms present during the same 2-week period, where this represents a change from previous functioning; and where at least one of the symptoms is either depressed mood or loss of interest or pleasure. Symptoms of a depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death, recurrent suicidal ideation without a specific plan, or a suicide attempt or a specific plan for committing suicide. Further, the symptoms cause clinically significant distress or impairment in social, occupational, or other important areas of functioning. (Diagnostic and Statistical Manual of Mental Disorders, 4^{th} Edition, American Psychiatric Association, 1994).

Dysthymia is defined as a mood disorder characterized by chronic depressed mood for a period of at least 2 years. Dysthymia can have a persistent or intermittent course and the depressed mood occurs for most of the day, for more days than not, and for at least 2 years. (Diagnostic and Statistical Manual of Mental Disorders, 4^{th} Edition, American Psychiatric Association, 1994).

Bipolar disorders, are characterized by unpredictable swings in mood between mania and depression (bipolar I disorder) or between hypomania and depression (bipolar II disorder) (Diagnostic and Statistical Manual of Mental Disorders, 4^{th} Edition, American Psychiatric Association, 1994).

Current pharmacological treatment options for unipolar depression include monotherapy or combination therapy with various classes of drugs including mono-amine oxidase inhibitors, tricyclic and heterocyclic antidepressants, selective serotonin reuptake inhibitors (SSRIs), selective serotonin and noradrenaline reuptake inhibitors (SSNRIs), noradrenergic and/or serotonergic agents, “natural products” (such as Kava-Kava, St. John's Wort), dietary supplement (such as s-adenosylmethionine) and others. Examples of pharmaceutical agents suitable for the treatment of depression include, but are not limited to imipramine, amitriptyline, desipramine, nortriptyline, doxepin, protriptyline, trimipramine, maprotiline, amoxapine, trazodone, bupropion, chlomipramine, fluoxetine, citalopram, sertraline, paroxetine, fluvoxamine, nefazadone, venlafaxine, reboxetine, mirtazapine, pheneizine, tranylcypromine, and/or moclobemide (eg, J. M. KENT, Lancet 2000, 355, 911-918; J. W. WILLIAMS JR, C. D. MULROW, E. CHIQUETTE, P. H. NOEL, C. AGUILAR, and J. CORNELL, Ann. Intern. Med. 2000, 132, 743-756; P. J. AMBROSINI, Psychiatr. Serv. 2000, 51, 627-633). Several of these agents including, but not limited to, serotonin reuptake inhibitors are also used when depression and anxiety co-exist, such as in anxious depression (R. B. LYDIARD and O. BRAWMAN-MINTZER, J. Clin. Psychiatry 1998, 59, Suppl. 18, 10-17; F. ROUILLON, Eur. Neuropsychopharmacol. 1999, 9 Suppl. 3, S87-S92).

In the clinic, 40-50% of depressed patients who are initially prescribed antidepressant therapy do not experience a timely remission of depression symptoms. This group typifies treatment-refractory depression, that is, a failure to demonstrate an “adequate” response to an “adequate” treatment trial (that is, sufficient intensity of treatment for sufficient duration) (R. M. BERMAN, M. NARASIMHAN, and D. S. CHARNEY, Depress. Anxiety 1997, 5, 154-164). Moreover, about 20-30% of depressed patients remain partially or totally resistant to pharmacological treatment including combination treatments (J. ANANTH, Psychother. Psychosom. 1998, 67, 61-70; R. J. CADIEUX, Am. Fam. Physician 1998, 58, 2059-2062). Increasingly, treatment of resistant depression includes augmentation strategies including treatment with pharmacological agents such as, lithium, carbamazepine, and triiodothyronine, and the like (M. HATZINGER and E. HOLSBOER-TRACHSLER, Wien. Med. Wochenschr. 1999, 149, 511-514; C. B. NEMEROFF, Depress. Anxiety 1996-1997, 4, 169-181; T. A. KETTER, R. M. POST, P. I. PAREKH and K. WORTHINGTON, J. Clin. Psychiatry 1995, 56, 471-475; R. T. JOFFE, W. SINGER, A. J. LEVITT, C. MACDONALD, Arch. Gen. Psychiatry 1993, 50, 397-393).

Antidepressant use in bipolar disorder is generally, intentionally restricted to avoid the risk of mania and the risk of rapid cycling induced by antidepressants in bipolar disorder (H. J. MOLLER and H. GRUNZE, Eur. Arch. Psychiatry Clin. Neurosci. 2000, 250, 57-68; J. R. CALABRESE, D. J. RAPPORT, S. E. KIMMEL, and M. D. SHELTON, Eur. Neuropsychopharmacol. 1999, 9, S109-S112). Moreover, none of the mood stabilizers used in bipolar disorder have proven antidepressive efficacy (H. J. MOLLER and H. GRUNZE, Eur. Arch. Psychiatry Clin. Neurosci. 2000, 250, 57-68).

There remains a need to provide an effective treatment for depression.

### SUMMARY OF THE INVENTION

The present invention is directed to a method for the treatment of depression comprising administering to a subject in need thereof a therapeutically effective amount of a compound of formula (I)

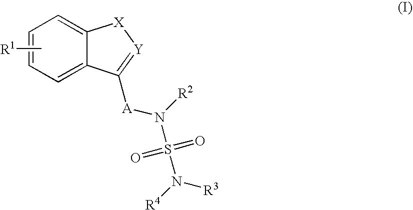

wherein

R¹ is selected from the group consisting of hydrogen, halogen, hydroxy, methoxy, trifluoromethyl, nitro and cyano;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and C₁₋₄alkyl;

alternatively, R³ and R⁴ are taken together with the nitrogen atom to which they are bound to form a 5 to 7 membered, saturated, partially unsaturated or aromatic ring structure, optionally containing one to three additional heteroatoms independently selected from the group consisting of O, N and S;

or a pharmaceutically acceptable salt thereof.

The present invention is further directed to a method for the treatment of depression comprising administering to a subject in need thereof co-therapy with a therapeutically effective amount of at least one antidepressant and a compound of formula (I) as herein defined.

Exemplifying the invention is a method of treating major depressive disorder, unipolar depression, treatment refractory depression, resistant depression, anxious depression or dysthymia comprising administering to a subject in need thereof a therapeutically effective amount of any of the compounds or pharmaceutical compositions described above.

In another example, the present invention is directed to a method of treating major depressive disorder, unipolar depression, treatment refractory depression, resistant depression, anxious depression or dysthymia comprising administering to a subject in need thereof at least one antidepressant in combination with any of the compounds or pharmaceutical compositions described above.

### BRIEF DESCRIPTION OF THE FIGURES

FIG. 1: Charts (A)-(E) Illustrate the time on feeder for paired submissive versus dominant rats.

Chart (A): Submissive rats were treated with Compound #1 at 6 mg/kg/day BID; dominant rats were treated with vehicle BID.

Chart (B): Submissive rate were treated with Compound #1 at 30 mg/kg/day BID; dominant rats were treated with vehicle BID.

Chart (C): Submissive rats were treated with Compound #1 at 60 mg/kg/day BID; dominant rats were treated with vehicle BID.

Chart (D): Submissive rats were treated with fluoxetine at 10 mg/kg/day QD; dominant rats were treated with water QD.

Chart (E): Submissive and dominant rats were treated with vehicle.

### DETAILED DESCRIPTION OF THE INVENTION

The present invention is directed to a method for the treatment of depression comprising administering to a subject in need thereof a therapeutically effective amount of a compound of formula (I)

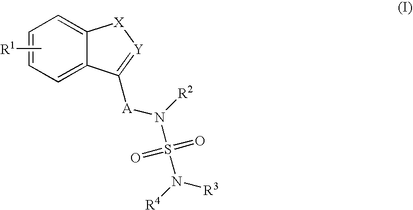

or a pharmaceutically acceptable salt thereof, wherein R¹, R², R³, R⁴, —X—Y— and A are as herein defined. The present invention is further directed to the treatment of depression comprising administering to a subject in need thereof a therapeutically effective amount of a compound of formula (I) in combination with at least one antidepressant.

As used herein, the term “depression” shall be defined to include major depressive disorder, unipolar depression, treatment-refractory depression, resistant depression, anxious depression and dysthymia (also referred to as dysthymic disorder). Preferably, the depression is major depressive disorder, unipolar depression, treatment-refractory depression, resistant depression or anxious depression. More preferably, the depression is major depressive disorder.

As used herein, unless otherwise noted, the term “antidepressant” shall mean any pharmaceutical agent which treats depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, and the like; serotonin receptor antagonists such as nefazadone, and the like; serotonin noradrenergic reuptake inhibitors such as venlafaxine, milnacipran and the like; noradrenergic and specific serotonergic agents such as mirtazapine, and the like; noradrenaline reuptake inhibitors such as reboxetine, and the like; atypical antidepressants such as bupropion, and the like; natural products such as Kava-Kava, St. John's Wort, and the like; dietary supplements such as s-adenosylmethionine, and the like; and neuropeptides such as thyrotropin-releasing hormone and the like, and the like; compounds targeting neuropeptide receptors such as neurokinin receptor antagonists and the like; and hormones such as triiodothyronine, and the like. Preferably, the antidepressant is selected from the group consisting of fluoxetine, imipramine, bupropion, venlafaxine and sertaline.

One skilled in the art would be able to readily determined recommended dosage levels for known and/or marketed antidepressant and antipsychotic drugs by consulting appropriate references such as drug package inserts, FDA guidelines, the Physician's Desk Reference, and the like.

The term “subject” as used herein, refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

Wherein the present invention is directed to co-therapy or combination therapy, comprising administration of one or more compound(s) of formula (I) and one or more antidepressants, “therapeutically effective amount” shall mean that amount of the combination of agents taken together so that the combined effect elicits the desired biological or medicinal response. For example, the therapeutically effective amount of co-therapy comprising administration of a compound of formula (I) and at least on antidepressant would be the amount of the compound of formula (I) and the amount of the antidepressant that when taken together or sequentially have a combined effect that is therapeutically effective. Further, it will be recognized by one skilled in the art that in the case of co-therapy with a therapeutically effective amount, as in the example above, the amount of the compound of formula (I) and/or the amount of the antidepressant individually may or may not be therapeutically effective.

As used herein, the terms “co-therapy” and “combination therapy” shall mean treatment of a subject in need thereof by administering one or more compounds of formula (I) in combination with one or more antidepressant(s), wherein the compound(s) of formula (I) and the antidepressant(s) are administered by any suitable means, simultaneously, sequentially, separately or in a single pharmaceutical formulation. Where the compound(s) of formula (I) and the antidepressant(s) are administered in separate dosage forms, the number of dosages administered per day for each compound may be the same or different. The compound(s) of formula (I) and the antidepressant(s) may be administered via the same or different routes of administration. Examples of suitable methods of administration include, but are not limited to, oral, intravenous (iv), intramuscular (im), subcutaneous (sc), transdermal, and rectal. Compounds may also be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices. The compound(s) of formula (I) and the antidepressant(s) may be administered according to simultaneous or alternating regimens, at the same or different times during the course of the therapy, concurrently in divided or single forms.

In an embodiment of the present invention is a method for the treatment of depression comprising administering to a subject in need thereof a combination of one or more compounds of formula (I) with one or more compounds selected from the group consisting of mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, fluvoxamine, escitalopram oxalate, and the like; serotonin receptor antagonists such as nefazadone, and the like; serotonin noradrenergic reuptake inhibitors such as venlafaxine, milnacipran, duloxetine, and the like; noradrenergic and specific serotonergic agents such as mirtazapine, and the like; noradrenaline reuptake inhibitors such as reboxetine, and the like; atypical antidepressants such as bupropion, and the like; natural products such as Kava-Kava, St. John's Wort, and the like; dietary supplements such as s-adenosylmethionine, and the like; and neuropeptides such as thyrotropin-releasing hormone and the like, and the like; compounds targeting neuropeptide receptors such as neurokinin receptor antagonists and the like; and hormones such as triiodothyronine, and the like.

In an embodiment of the present invention is a method for the treatment of depression comprising administering to a subject in need thereof a combination of one or more compounds of formula (I) with one or more compounds selected from the group consisting of mono-amine oxidase inhibitors; tricyclics; tetracyclics; non-cyclics; triazolopyridines; serotonin reuptake inhibitors; serotonin receptor antagonists; serotonin noradrenergic reuptake inhibitors; serotonin noradrenergic reuptake inhibitors; noradrenergic and specific serotonergic agents; noradrenaline reuptake inhibitors; atypical antidepressants; natural products; dietary supplements; neuropeptides; compounds targeting neuropeptide receptors; and hormones.

Preferably, one or more compounds of formula (I) are administered in combination with one or more compounds selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

More preferably, one or more compounds of formula (I) are administered in combination with one or more compounds selected from the group consisting of mono-amino oxidase inhibitors, tricyclics and serotonin reuptake inhibitors.

Most preferably, one or more compounds of formula (I) are administered in combination with one or more compounds selected from the group consisting of serotonin reuptake inhibitors.

In an embodiment of the present invention is a method for the treatment of depression comprising administering to a subject in need thereof a combination of one or more compounds of formula (I) with one or more compounds selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, duloxetine, mirtazapine, bupropion, thyrotropin-releasing hormone and triiodothyronine.

Preferably, one or more compounds of formula (I) are administered in combination with one or more compounds selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

More preferably, one or more compounds of formula (I) are administered in combination with one or more compounds selected from the group consisting of phenelzine, tranylcypromine, moclobemide, imipramine, amitriptyline, desipramine, nortiptyline, doxepin, protriptyline, trimipramine, chlomipramine, amoxapine, fluoxetine, sertraline, paroxetine, citalopram, escitalopram and fluvoxamine.

Most preferably, one or more compounds of formula (I) are administered in combination with one or more compounds selected from the group consisting of fluoxetine, sertraline, paroxetine, citalopram and fluvoxamine.

In an embodiment of the present invention, is a method for the treatment of depression comprising administering to a subject in need thereof a combination of one or more compounds of formula (I) with one or more compounds selected from the group consisting of neuropeptides such as thyrotropin-releasing hormone and the like; compounds targeting neuropeptide receptors such as neurokinin receptors antagonists and the like; and hormones such as triiodothyronine and the like.

In an embodiment of the present invention, the compound of formula (I) is selected from the group wherein

R¹ is selected from the group consisting of hydrogen, halogen, hydroxy, methoxy, trifluoromethyl, nitro and cyano;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and methyl;

alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;

or a pharmaceutically acceptable salt thereof.

In another embodiment of the present invention, the compound of formula (I) is selected from the group wherein

R¹ is selected from the group consisting of hydrogen and halogen;

X—Y is selected from the group consisting of —S—CH—, —S—C(CH₃)—, —O—CH—, —O—C(CH₃)—, —N(CH₃)—CH— and —CH═CH—CH—;

A is selected from the group consisting of —CH₂— and —CH(CH₃)—;

R² is selected from the group consisting of hydrogen and methyl;

R³ and R⁴ are each independently selected from the group consisting of hydrogen and methyl;

and pharmaceutically acceptable salts thereof.

In another embodiment of the present invention, the compound of formula (I) is selected from the group wherein

R¹ is selected from the group consisting of hydrogen and halogen; wherein the halogen is bound at the 4-, 5- or 7-position;

X—Y is selected from the groups consisting of —O—CH—, —O—C(CH₃)—, —S—CH—, —S—C(CH₃)—, —N(CH₃)—CH— and —CH═CH—CH—;

A is selected from the group consisting of —CH₂— and —CH(CH₃)—;

R² is hydrogen;

R³ and R⁴ are each hydrogen;

and pharmaceutically acceptable salts thereof.

In another embodiment of the present invention, the compound of formula (I) is selected from the group wherein

R¹ is hydrogen;

X—Y is selected from the groups consisting of —O—CH—, —O—C(CH₃)—, —S—CH—, —S—C(CH₃)—, —N(CH₃)—CH— and —CH═CH—CH—;

A is selected from the group consisting of —CH₂— and —CH(CH₃)—;

R² is hydrogen;

R³ and R⁴ are each hydrogen;

and pharmaceutically acceptable salts thereof.

In another embodiment of the present invention, the compound of formula (I) is selected from the group wherein

R¹ is selected from the group consisting of hydrogen halogen, hydroxy, methoxy, trifluoromethyl, nitro and cyano; preferably, R¹ is selected from the group consisting of hydrogen and halogen; more preferably, R¹ is selected from the group consisting of hydrogen and halogen, wherein the halogen is bound at the 4-, 5- or 7-position;

X—Y is —S—CH—;

A is selected from the group consisting of —CH₂— and —CH(CH₃)—;

R² is selected from the group consisting of hydrogen and methyl; preferably, R² is hydrogen;

R³ and R⁴ are each independently selected from the group consisting of hydrogen and halogen; preferably, R³ and R⁴ are each hydrogen;

and pharmaceutically acceptable salts thereof.

In an embodiment of the present invention R¹ is selected from the group consisting of hydrogen, chloro, fluoro and bromo. In another embodiment of the present invention, the R¹ group is other than hydrogen and bound at the 4-, 5- or 7-position, preferably at the 5-position. In yet another embodiment of the present invention, the R¹ group is other than hydrogen and bound at the 5-, 6- or 8-position, preferably at the 6-position. In yet another embodiment of the present invention, R¹ is selected from the group consisting of hydrogen and halogen. In yet another embodiment of the present invention, R¹ is selected from the group consisting of hydroxy and methoxy. In yet another embodiment of the present invention, R¹ is selected from the group consisting of hydrogen, halogen and trifluoromethyl. In yet another embodiment of the present invention, R¹ is selected from the group consisting of hydrogen, halogen, trifluoromethyl, cyano and nitro. In yet another embodiment of the present invention, R¹ is selected from the group consisting of hydrogen, halogen, trifluoromethyl and cyano. In yet another embodiment of the present invention, R¹ is selected from the group consisting of trifluoromethyl and cyano. In yet another embodiment of the present invention, R¹ is selected from the group consisting of hydrogen, 4-bromo, 5-chloro, 5-fluoro, 5-bromo, 5-trifluoromethyl-5-cyano and 7-cyano.

In an embodiment of the present invention R² is hydrogen. In another embodiment of the present invention R³ and R⁴ are each hydrogen. In yet another embodiment of the present invention R² is hydrogen, R³ is hydrogen and R⁴ is hydrogen.

In an embodiment of the present invention, R³ and R⁴ are each independently selected from the group consisting of hydrogen and C₁₋₄alkyl. In another embodiment of the present invention, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S.

In an embodiment of the present invention, R³ and R⁴ are each independently selected from the group consisting of hydrogen, methyl and ethyl. In another embodiment of the present invention, R³ and R⁴ are each independently selected from the group consisting of hydrogen and methyl. In yet another embodiment of the present invention, R³ and R⁴ are each independently selected from the group consisting of hydrogen and ethyl. In yet another embodiment of the present invention, R³ is hydrogen and R⁴ is ethyl.

In an embodiment of the present invention R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, S and N. In another embodiment of the present invention R³ and R⁴ are taken together with the nitrogen atom to which they are bound to form a 5 to 7 membered saturated ring structure, optionally containing one to two additional heteroatoms independently selected from the group consisting of O, S and N. In another embodiment of the present invention R³ and R⁴ are taken together with the nitrogen atom to which they are bound to form a 5 to 7 membered aromatic ring structure, optionally containing one to two additional heteroatoms independently selected from the group consisting of O, S and N.

Preferably, R³ and R⁴ are taken together with the nitrogen atom to which they are bound to form a 5 to 6 membered saturated, partially unsaturated or aromatic ring structure, optionally containing one to two additional heteroatoms independently selected from the group consisting of O, S and N. More preferably, R³ and R⁴ are taken together with the nitrogen atom to which they are bound to form a 6 membered saturated, partially unsaturated or aromatic ring structure, optionally containing one to two additional heteroatoms independently selected from the group consisting of O, S and N.

Preferably, R³ and R⁴ are taken together with the nitrogen atom to which they are bound to form a 5 to 7 (more preferably 5 to 6) membered saturated or aromatic ring structure, optionally containing one to two (preferably one) additional heteroatoms independently selected from the group consisting of O, S and N (preferably O or N, more preferably N).

In another embodiment of the present invention, R³ and R⁴ are taken together with the nitrogen atom to which they are bound to form a 5 to 6 membered saturated or aromatic ring structure, optionally containing one to two (preferably one) additional heteroatoms independently selected from the group consisting of O, S and N (preferably O or N, more preferably, N).

Preferably, the 5 to 7 membered saturated, partially unsaturated or aromatic ring structure contains 0 to 1 additional heteroatoms independently selected from the group consisting of O, S and N. Preferably, the heteroatom is independently selected from the group consisting of O and N, more preferably, the heteroatom is N.

Suitable examples of the 5 to 7 membered, saturated, partially unsaturated or aromatic ring structures which optionally contain one to two additional heteroatoms independently selected from the group consisting of O, S and N include, but are not limited to pyrrolyl, pyrrolidinyl, pyrrolinyl, morpholinyl, piperidinyl, piperazinyl, imidazolyl, pyrazolyl, pyridyl, imidazolyl, thiomorpholinyl, pyrazinyl, triazinyl, azepinyl, and the like. Preferred 5 to 7 membered, saturated, partially unsaturated or aromatic ring structures which optional containing one to two additional heteroatoms independently selected from the group consisting of O, S and N include, but are not limited, to imidazolyl, pyrrolidinyl, piperidinyl and morpholinyl.

In an embodiment of the present invention A is —CH₂—.

In an embodiment of the present invention X—Y is selected from the group consisting of —S—CH—, —O—CH—, —O—C(CH₃)—, —N(CH₃)—CH— and —CH═CH—CH—. In another embodiment of the present invention X—Y is selected from the group consisting of —S—CH—, —O—CH—, —O—C(CH₃)— and —CH═CH—CH—. In yet another embodiment of the present invention X—Y is selected form the group consisting of —S—CH—, —O—CH—, —O—C(CH₃)— and —N(CH₃)—CH—. In yet another embodiment of the present invention X—Y is selected from the group consisting of —S—CH—, —O—CH—, —N(CH₃)—CH— and —CH═CH—CH—. In yet another embodiment of the present invention X—Y is selected from the group consisting of —S—CH—, —O—CH— and —CH═CH—C—. In yet another embodiment of the present invention, X—Y is selected from the group consisting of —S—CH— and —O—CH—. In yet another embodiment of the present invention, X—Y is selected from the group consisting of S—CH—, —S—C(CH₃)—, —O—CH—, —O—C(CH₃)— and —N(CH₃)—CH—.

In an embodiment of the present invention, X— is —S—CH—. In another embodiment of the present invention X—Y is —CH═CH═CH—. In yet another embodiment of the present invention X—Y is —N(CH₃)—CH—. In yet another embodiment of the present invention X—Y is selected from the group consisting of —O—CH— and —O—C(CH₃)—.

In an embodiment, the present invention is directed to a compounds selected from the group consisting of N-(benzo[b]thien-3-ylmethyl)-sulfamide; N-[(5-chlorobenzo[b]thien-3-yl)methyl]-sulfamide; N-(3-benzofuranyl methyl)-sulfamide; N-[(5-fluorobenzo[b]thien-3-yl)methyl]-sulfamide; N-(1-benzo[b]thien-3-ylethyl)-sulfamide; N-(1-naphthalenylmethyl)-sulfamide; N-[(2-methyl-3-benzofuranyl)methyl]-sulfamide; N-[(5-bromobenzo[b]thien-3-yl)methyl]-sulfamide; N-[(4-bromobenzo[b]thien-3-yl)methyl]-sulfamide; N-[(7-fluorobenzo[b]thien-3-yl)methyl]-sulfamide; N-[(1-methyl-1H-indol-3-yl)methyl]-sulfamide; N-[(4-trifluoromethylbenzo[b]thien-3-yl)methyl]-sulfamide; N-[(4-cyanobenzo[b]thien-3-yl)methyl]-sulfamide; N-[(benzo[b]thien-3-yl)methyl]-sulfamoylpyrrolidine; N-[(benzo[b]thien-3-yl)methyl]-N′-ethylsulfamide; Imidazole-1-sulfonic acid [(benzo[b]thien-3-yl)methyl]-amide; and pharmaceutically acceptable salts thereof.

Additional embodiments of the present invention, include those wherein the substituents selected for one or more of the variables defined herein (i.e. R¹, R², R³, R⁴, X—Y and A) are independently selected to be any individual substituent or any subset of substituents selected from the complete list as defined herein.

Representative compounds useful in the treatment of depression are as listed in Table 1 and 2, below.

TABLE 1
Representative Compounds of Formula (I)

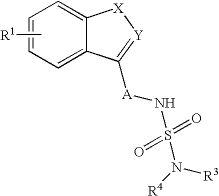

| ID No. | R¹ | —X—Y— | A | R³ | R⁴ |
|---|---|---|---|---|---|
| 1 | H | —S—CH— | —CH₂— | H | H |
| 3 | 5-Cl | —S—CH— | —CH₂— | H | H |
| 6 | H | —O—CH— | —CH₂— | H | H |
| 7 | H | —N(CH₃)—CH— | —CH₂— | H | H |
| 8 | 5-F | —S—CH— | —CH₂— | H | H |
| 9 | H | —S—CH— | —CH(CH₃)— | H | H |
| 10 | H | —CH═CH—CH— | —CH₂— | H | H |
| 13 | H | —O—C(CH₃) | —CH₂— | H | H |
| 15 | 5-Br | —S—CH— | —CH₂— | H | H |
| 17 | 4-Br | —S—CH— | —CH₂— | H | H |
| 18 | 7-F | —S—CH— | —CH₂— | H | H |
| 19 | 5-CF₃ | —S—CH— | —CH₂— | H | H |
| 20 | 5-CN | —S—CH— | —CH₂— | H | H |
| 21 | H | —S—CH— | —CH₂— | H | ethyl |

TABLE 2

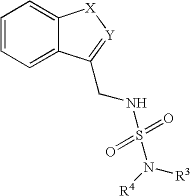

| ID No. | —X—Y— | R3 + R4 together with the N atom |
|---|---|---|
| 101 | —S—CH— | N-pyrrolidinyl |
| 102 | —S—CH— | N-imidazolyl |

As used herein, “halogen” shall mean chlorine, bromine, fluorine and iodine.

As used herein, the term “alkyl” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “C₁₋₄alkyl” means a carbon chain composition of 1-4 carbon atoms.

When a particular group is “substituted” (e.g., alkyl, phenyl, aryl, heteroalkyl, heteroaryl), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

Unless otherwise noted, the position at which the R¹ substituent is bound will be determined by counting around the core structure in a clockwise manner beginning at the X—Y positions as 1, 2 and continuing from thereon as follows:

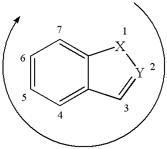

Should the X—Y substituent be —CH═CH—CH—, then the X—Y group will be counted as 1, 2, 3 and counting then continued clockwise around the core structure as previously noted.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

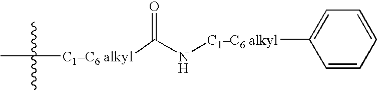

Abbreviations used in the specification, particularly the Schemes and Examples, are as follows:
DCE=Dichloroethane
DCM=Dichloromethane
DMF=N,N-Dimethylformamide
DMSO=Dimethylsulfoxide
LAH=Lithium Aluminum Hydride
MTBE=Methyl-tert-butyl ether
THF=Tetrahydrofuran
TLC=Thin Layer Chromatography

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following:

acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following:

acids including acetic acid, 2,2-dichlorolactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (−)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitric acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and

bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) wherein A is —CH₂— may be prepared according to the process outlined in Scheme 1.

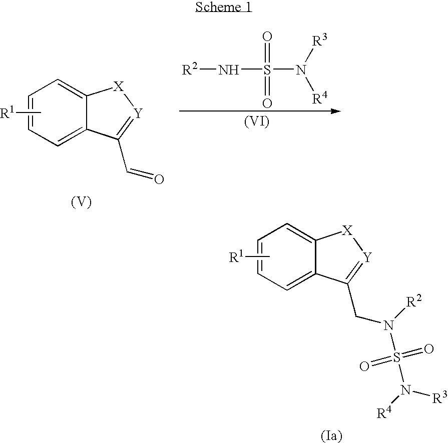

Accordingly, a suitably substituted compound of formula (V), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as ethanol, methanol, dioxane, and the like, preferably, in an anhydrous organic solvent, preferably, at an elevated temperature in the range of about 50° C. to about 100° C., more preferably at about reflux temperature, to yield the corresponding compound of formula (Ia).

Compounds of formula (I) may alternatively be prepared according to the process outlined in Scheme 2.

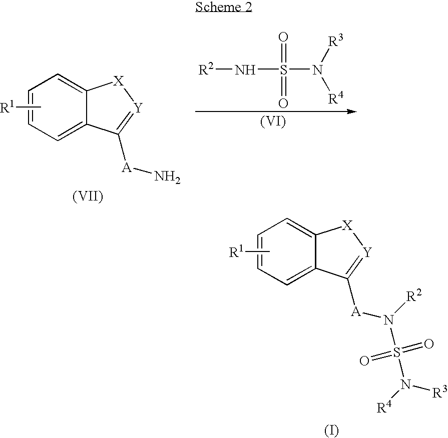

Accordingly, a suitably substituted compound of formula (VII), a known compound or compound prepared by known methods, is reacted with a suitably substituted compound of formula (VI), a known compound or compound prepared by known methods, wherein the compound of formula (VI) is present in an amount in the range of about 2 to about 5 equivalents, in an organic solvent such as THF, dioxane, and the like, preferably, in an anhydrous organic solvent, preferably, at an elevated temperature in the range of about 50° C. to about 100° C., more preferably at about reflux temperature, to yield the corresponding compound of formula (I).

Compounds of formula (VII) wherein A is —CH₂— may, for example, be prepared by according to the process outlined in Scheme 3.

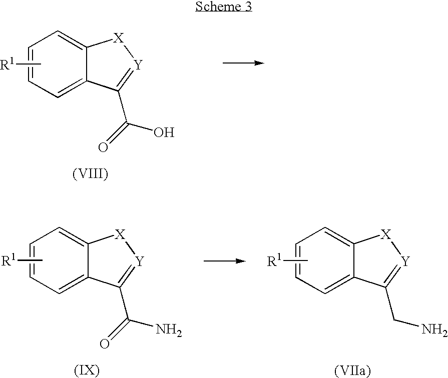

Accordingly, a suitably substituted a compound of formula (VIII), a known compound or compound prepared by known methods is reacted with an activating agent such as oxalyl chloride, sulfonyl chloride, and the like, and then reacted with an amine source such as ammonia, ammonium hydroxide, and the like, in an organic solvent such as THF, diethyl ether, DCM, DCE, and the like, to yield the corresponding compound of formula (IX).

The compound of formula (IX) is reacted with a suitably selected reducing agent such as LAH, borane, and the like, in an organic solvent such as THF, diethyl ether, and the like, to yield the corresponding compound of formula (VIIa).

Compounds of formula (VII) wherein A is —CH(CH₃)— may, for example, be prepared according to the process outlined in Scheme 4.

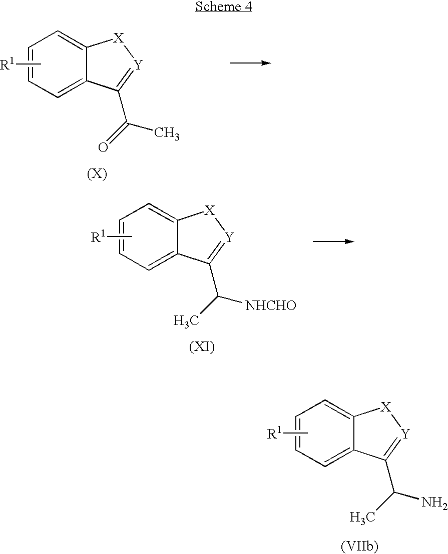

Accordingly, a suitably substituted compounds of formula (X), a known compound or compound prepared by known methods, is reacted with a mixture of formamide and formic acid, wherein the mixture of formamide and formic acid is present in an amount greater than about 1 equivalent, preferably, in an excess amount of greater than about 5 equivalent, at an elevated temperature of about 150° C., to yield the corresponding compound of formula (XI).

The compound of formula (XI) is hydrolyzed by reacting with concentrated HCl, concentrated H₂SO₄, and the like, at an elevated temperature, preferably at reflux temperature, to yield the corresponding compound of formula (VIIb).

Compounds of formula (VII) may alternatively, be prepared according to the process outlined in Scheme 5.

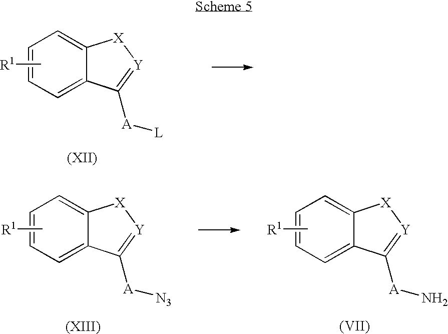

Accordingly, a suitably substituted compound of formula (XII), wherein L is a leaving group such as Br, Cl, I, tosylate, mesylate, and the like, a known compound or compound prepared by known methods, is reacted with sodium azide, in an organic solvent such a DMF, DMSO, methanol, ethanol, and the like, to yield the corresponding compound of formula (XIII).

The compound of formula (XIII) is reacted with a suitably selected reducing agent such as LAH, triphenylphosphine, H_{2(g)}, and the like, according to known methods, to yield the corresponding compound of formula (VII).

Compounds of formula (VII) wherein A is CH₂ and X—Y is —O—CH₂— may, for example, be prepared according to the process outlined in Scheme 6.

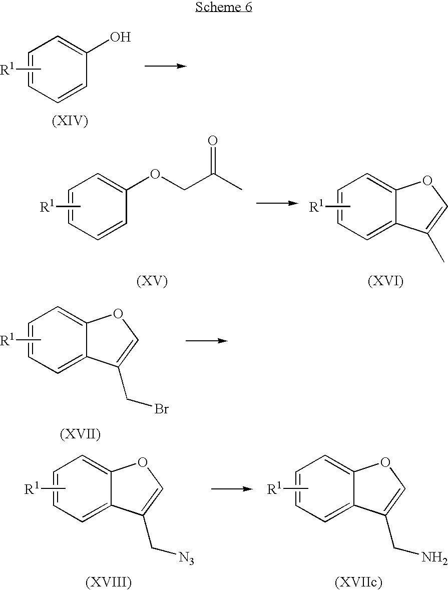

Accordingly, a suitably substituted phenol, a compound of formula (XIV), a known compound or compound prepared by known methods is reacted with bromoacetone, a known compound, in the presence of a base such as K₂CO₃, Na₂CO₃, NaH, triethylamine, pyridine, and the like, in an organic solvent such as acetonitrile, DMF, THF, and the like, optionally at an elevated temperature, to yield the corresponding compound of formula (XV).

The compound of formula (XV) is reacted with an acid such as polyphosphoric acid, sulfuric acid, hydrochloric acid, and the like, preferably with polyphosphoric acid, preferably in the absence of a solvent (one skilled in the art will recognize that the polyphosphoric acid acts as the solvent), to yield the corresponding compound of formula (XVI).

The compound of formula (XVI) is reacted with a source of bromine such as N-bromosuccinimide in the presence of benzoylperoixde, Br₂, and the like, in an organic solvent such as carbon tetrachloride, chloroform, DCM, and the like, preferably in a halogenated organic solvent, to yield the corresponding compound of formula (XVII).

The compound of formula (XVII) is reacted with sodium azide, in an organic solvent such a DMF, DMSO, methanol, ethanol, and the like, to yield the corresponding compound of formula (XVIII).

The compound of formula (XVI II) is reacted with a suitably selected reducing agent such as LAH, triphenylphosphine, H_{2(g)}, and the like, according to known methods, to yield the corresponding compound of formula (VIIc).

Compounds of formula (V) wherein X—Y is —S—CH— may, for example, be prepared according to the process outlined in Scheme 7.

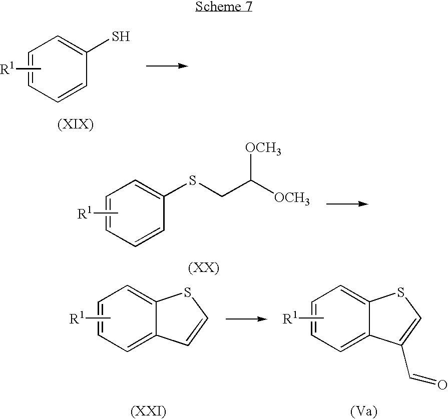

Accordingly, a suitably substituted compound of formula (XIX), a known compound or compound prepared by known methods is reacted with choroacetaldehyde dimethyl acetal or bromoacetaldehyde dimethyl acetal, a known compound, in the presence of a base such as potassium-tert-butoxide, sodium-tert-butxide, potassium carbonate, potassium hydroxide, and the like, in an organic solvent such as THF, DMF, acetonitrile, and the like, to yield the corresponding compound of formula (XX).

The compound of formula (XX) is reacted with reacted with an acid such as polyphosphoric acid, sulfuric acid, hydrochloric acid, and the like, preferably with polyphosphoric acid in the presence of chlorobenzene, preferably in the absence of a solvent (one skilled in the art will recognize that the polyphosphoric acid and/or the chlorobenzene may act as the solvent), at an elevated temperature in the range of from about 100 to 200° C., preferably at an elevated temperature of about reflux temperature, to yield the corresponding compound of formula (XXI).

The compound of formula (XXI) is reacted with a formylating reagent such as dichloromethyl methyl ether, and the like, in the presence of Lewis acid catalyst such as titanium tetrachloride, aluminum trichloride, tin tetrachloride, and the like, in an organic solvent such as DCM, chloroform, and the like, at a temperature in the range of from about 0° C. to about room temperature, to yield the corresponding compound of formula (Va).

Compounds of formula (I) wherein R³ and/or R⁴ are other than hydrogen or R³ and R⁴ are taken together with the nitrogen to which they are bound to form a ring structure, may alternatively be prepared according to the process outlined in Scheme 8.

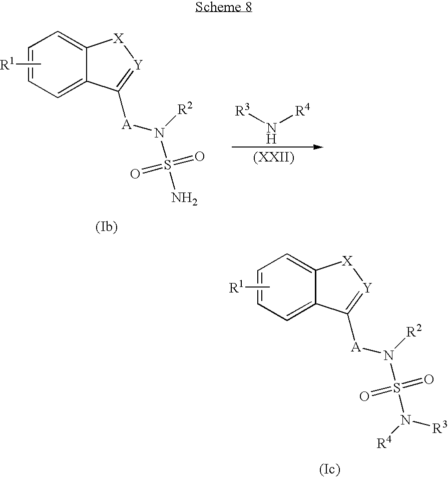

Accordingly, a suitably substituted compound of formula (Ib), is reacted with a suitably substituted amine, a compound of formula (XXII), a known compound or compound prepared by known methods, in water or an organic solvent such as dioxane, ethanol, THF, isopropanol, and the like, provide that the compound of formula (Ib) and the compound of formula (XXII) are at least partially soluble in the water or organic solvent, at a temperature in the range of from about room temperature to about reflux, preferably at about reflux temperature, to yield the corresponding compound of formula (Ic).

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1-1000 mg and may be given at a dosage of from about 0.01-200.0 mg/kg/day, preferably from about 0.1 to 100 mg/kg/day, more preferably from about 0.5-50 mg/kg/day, more preferably from about 1.0-25.0 mg/kg/day or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 1000 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating depression described in the present invention may also be carried out using a pharmaceutical composition comprising any of the compounds as defined herein and a pharmaceutically acceptable carrier. The pharmaceutical composition may contain between about 0.1 mg and 1000 mg, preferably about 50 to 500 mg, of the compound, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Compounds of this invention may be administered in any of the foregoing compositions and according to dosage regimens established in the art whenever treatment of depression is required.

The daily dosage of the products may be varied over a wide range from 0.01 to 200 mg/kg per adult human per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 200 mg/kg of body weight per day. Preferably, the range is from about 0.1 to about 100.0 mg/kg of body weight per day, more preferably, from about 0.5 mg/kg to about 50 mg/kg, more preferably, from about 1.0 to about 25.0 mg/kg of body weight per day. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

### EXAMPLE 1

### N-(benzorblthien-3-vimethyl)-sulfamide (Compound #1)

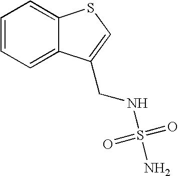

Thianaphthene-3-carboxaldehyde (1.62 g, 10.0 mmol) was dissolved in anhydrous ethanol (50 mL). Sulfamide (4.0 g, 42 mmol) was added and the mixture was heated to reflux for 16 hours. The mixture was cooled to room temperature. Sodium borohydride (0.416 g, 11.0 mmol) was added and the mixture was stirred at room temperature for three hours. The reaction was diluted with water (50 mL) and extracted with chloroform (3×75 mL). The extracts were concentrated and chromatographed (5% methanol in DCM) to yield the title compound as a white solid.

¹H NMR (DMSO-d₆): δ 7.98 (1H, dd, J=6.5, 2.3 Hz), 7.92 (1H, dd, J=6.6, 2.4 Hz), 7.62 (1H, s), 7.36-7.45 (2H, m), 7.08 (1H, t, J=6.3 Hz), 6.72 (2H, s), 4.31 (2H, d, J=6.3 Hz).

### EXAMPLE 2

### N-[(5-chlorobenzorblthien-3-yl)methyl]-sulfamide (Compound #3)

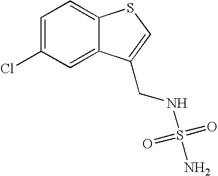

(5-Chloro-1-benzothiophene-3-yl)methylamine (0.820 g, 4.15 mmol) and sulfamide (2.5 g, 26 mmol) were combined in anhydrous dioxane (50 mL) and the mixture was heated to reflux for four hours. The reaction was cooled and diluted with water (50 mL). The solution was extracted with chloroform (3×75 mL). The extracts were concentrated and chromatographed (5% methanol in DCM) to yield the title compound as a white solid.

¹H NMR (DMSO-d₆): δ 8.05 (2H, m), 7.74 (1H, s), 7.40 (1H, d, J=6.5 Hz), 7.07 (1H, t, J=6.3 Hz), 6.72 (2H, s), 4.26 (2H, d, J=6.4 Hz).

### EXAMPLE 3

### N-[(1-methyl-1H-indol-3-yl)methyl]-sulfamide (Compound #7)

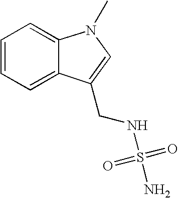

N-Methylindole-3-carboxaldehyde (1.66 g, 10.4 mmol) was dissolved in anhydrous ethanol (50 mL). Sulfamide (4.5 g, 47 mmol) was added and the mixture was heated to reflux for 16 hours. Additional sulfamide (1.0 g, 10.4 mmol) was added and the mixture was heated to reflux for 24 hours. The mixture was cooled to room temperature. Sodium borohydride (0.722 g, 12.5 mmol) was added and the mixture was stirred at room temperature for one hour. The reaction was diluted with water (50 mL) and extracted with DCM (3×75 mL). The extracts were concentrated and about 1 mL of methanol was added to create a slurry which was filtered to yield the title compound as a white powder.

¹H NMR (CD₃OD): δ 7.67 (1H, d, J=5.9 Hz), 7.32 (1H, d, J=6.2 Hz), 7.14-7.19 (2H, m), 7.06 (1H, dt, J=7.7, 0.7 Hz), 4.36 (2H, s), 3.75 (3H, s)

MS (M−H)⁻ 237.6.

### EXAMPLE 4

### N-(3-benzofuranylmethyl)-sulfamide (Compound #6)

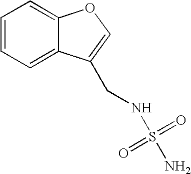

Benzofuran-3-carboxylic acid (1.91 g, 11.8 mmol) was suspended in anhydrous DCM (75 mL). Oxalyl chloride (2.0 M in DCM, 6.48 mL) and then one drop of dimethylformamide were added. The solution was stirred at room temperature for two hours, then ammonium hydroxide (concentrated, 10 mL) was added. The resulting mixture was diluted with water (100 mL) and extracted with DCM (3×100 mL). The extracts were concentrated to a gray solid and dissolved in anhydrous THF (100 mL). Lithium aluminum hydride (1.0 M in THF, 11.8 mL) was added. The mixture was stirred at room temperature for 16 hours. A minimal amount of saturated aqueous NaHCO₃ and then MgSO₄ were added. The mixture was filtered and then extracted with 1 N HCl. The aqueous extracts were adjusted to pH 14 with 3N NaOH and extracted with DCM. The organic extracts were dried with magnesium sulfate and concentrated to a colorless oil. The oil was dissolved in dioxane (50 mL) and sulfamide (3.7 g, 38 mmol) was added. The mixture was heated to reflux for 4 hours, cooled to room temperature, and concentrated. The resulting solid was chromatographed (5% methanol in DCM) to yield the title compound as a slightly yellow solid.

¹H NMR (CD₃OD): δ 7.53 (1H, d, J=5.7 Hz), 7.44 (1H, d, J=6.0 Hz), 7.16-7.26 (2H, m), 6.73 (1H, s), 4.35 (2H, s).

### EXAMPLE 5

### N-[(5-fluorobenzo[b]thien-3-yl)methyl]-sulfamide (Compound #8)

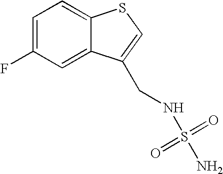

5-Fluoro-3-methylbenzothiophene (1.14 g, 6.83 mmol), benzoyl peroxide (0.165 g, 0.68 mmol) and N-bromosuccinimide (1.70 g, 7.52 mmol) were combined in carbon tetrachloride (25 mL) and the mixture was heated to reflux for 3 hours. The yellow solution was cooled, diluted with water, and extracted with DCM (2×50 mL). The extracts were washed with brine (100 mL), dried with magnesium sulfate, and concentrated to an orange solid. The solid was dissolved in anhydrous DMF. Sodium azide (4.0 g, 61 mmol) was added and the mixture was stirred for 16 hours at room temperature. The reaction was diluted with water (100 mL) and extracted with diethyl ether (2×75 mL). The extracts were washed with brine (100 mL), dried with magnesium sulfate, and concentrated to a yellow oil. The oil was dissolved in a mixture of THF (50 mL) and water (5 mL). Triphenylphosphine (3.60 g, 13.7 mmol) was added. The mixture was stirred at room temperature for 16 hours. The reaction was concentrated and chromatographed (2 to 5% methanol in DCM). The resulting C-(5-fluoro-benzo[b]thien-3-yl)-methylamine (1.04 g, 5.73 mmol) was dissolved in anhydrous dioxane (50 mL) and sulfamide (2.75 g, 28.7 mmol) was added. The reaction was heated to reflux for 4 hours, cooled to room temperature, and concentrated to a solid which was chromatographed (5% methanol in DCM) to yield the title compound as a white solid.

¹H NMR (CD₃OD): δ 7.85 (1H, dd, J=6.6, 3.6 Hz), 7.66 (1H, dd, J=7.4, 1.8 Hz), 7.62 (1H, s), 7.13-7.18 (1H, m), 4.40 (2H, s).

### EXAMPLE 6

### N-(1-benzo[b]thien-3-ylethyl)-sulfamide (Compound #9)

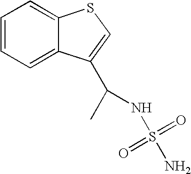

3-Acetylthianaphthene (3.00 g, 17.0 mmol) was added to a mixture of formic acid (10 mL) and formamide (10 mL). The solution was heated to 150° C. for 8 hours. The reaction was cooled to room temperature, diluted with water (50 mL), and extracted with diethyl ether (3×50 mL). The ether extracts were washed with saturated aqueous NaHCO₃ and brine. The solution was concentrated and chromatographed (5% methanol in DCM) to yield N-(1-benzo[b]thiophen-3-yl-ethyl)-formamide (1.76 g) as a white solid which was suspended in concentrated HCl (30 mL). The mixture was heated to reflux for 1.5 hours then diluted with water (100 mL). 3N NaOH was added until the pH was 14. The mixture was extracted with diethyl ether (3×100 mL) then dried with magnesium sulfate and concentrated to an orange oil. The oil was dissolved in anhydrous dioxane (75 mL) and sulfamide was added. The mixture was heated to reflux for 2 hours then diluted with water (50 ml). The solution was extracted with ethyl acetate (2×50 mL), dried with magnesium sulfate, concentrated, and chromatographed (2.5% to 5% methanol in DCM) to yield the title compound as a white solid.

¹H NMR (CD₃OD): δ 8.01 (1H, dd, J=5.5, 0.7 Hz), 7.85 (1H, dt, J=6.0, 0.6 Hz), 7.49 (1H, s), 7.31-7.40 (2H, m), 4.95 (1H, q, J=5.1 Hz), 1.67 (3H, d, J=5.1 Hz).

### EXAMPLE 7

### N-(1-naphthalenvimethyl)-sulfamide (Compound #10)

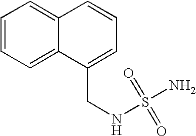

1-Naphthanlenemethylamine (2.00 g, 12.7 mmol) and sulfamide (5.0 g, 52 mmol) were combined in anhydrous dioxane (100 mL) and the mixture was heated to reflux for 6 hours. The reaction was cooled to room temperature and was filtered. The filtrate was concentrated to a solid and washed with water until TLC indicated no remaining trace of sulfamide in the solid. The collected solid was dried under vacuum to yield the title compound as a white solid.

¹H NMR (CDCl₃): δ 8.09 (1H, d, J=6.3 Hz), 7.86 (1H, dd, J=12.9, 6.2 Hz), 7.42-7.61 (4H, m), 4.75 (2H, d, J=4.4 Hz), 4.58 (1H, br s), 4.51 (2H, br s).

### EXAMPLE 8

### N-[(2-methyl-3-benzofuranyl)methyl]-sulfamide (Compound #13)

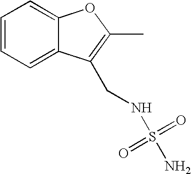

2-Methylbenzofuran-3-carbaldehyde (0.51 g, 3.18 mmol) was dissolved in anhydrous ethanol (25 mL). Sulfamide (1.5 g, 16 mmol) was added and the mixture was heated to reflux for 4 days. The mixture was cooled to room temperature. Sodium borohydride (0.132 g, 3.50 mmol) was added and the mixture was stirred at room temperature for 24 hours. The reaction was diluted with water (100 mL) and extracted with DCM (3×75 mL). The extracts were concentrated and suspended in a minimal amount of DCM and filtered to yield the title compound as a white solid.

¹H NMR (DMSO-d₆): δ 7.65 (1H, dd, J=6.4, 2.6 Hz), 7.43-7.47 (1H, m), 7.19-7.23 (2H, m), 6.87 (1H, t, J=6.2 Hz), 6.68 (2H, s), 4.11 (2H, d, J=6.2 Hz), 2.42 (3H, s).

### EXAMPLE 9

### N-[(5-bromobenzo[b]thien-3-yl)methyl]-sulfamide (Compound #15)

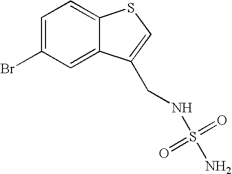

5-Bromobenzothiophene (1.60 g, 7.51 mmol) and dichloromethyl methyl ether (1.29 g, 11.3 mmol) were dissolved in anhydrous 1,2-dichloroethane (75 mL). Titanium tetrachloride (2.14 g, 11.3 mmol) was added, turning the solution dark. After one hour at room temperature, the reaction was poured into a mixture of saturated aqueous NaHCO₃ and ice. The mixture was stirred for about 30 minutes and then was extracted with DCM (2×100 mL). The extracts were concentrated and chromatographed (0 to 5% ethyl acetate in hexane) to yield 5-bromo-benzo[b]thiophene-3-carbaldehyde (1.32 g). The 5-bromobenzothiophene-3-carboxaldehyde (1.20 g, 4.98 mmol) and sulfamide (4.0 g, 42 mmol) were combined in anhydrous ethanol (25 mL) and heated to reflux for three days. The reaction was cooled to room temperature and sodium borohydride (0.207 g, 5.47 mmol) was added. After five hours, water (50 ml) was added and the solution was extracted with chloroform (3×50 mL). The extracts were concentrated, suspended in a minimal amount of DCM, and filtered to provide the title compound as a yellow solid.

¹H NMR (DMSO-d₆): δ 8.12 (1H, d, J=1.8 Hz), 7.97 (1H, d, J=8.6), 7.71 (1H, s), 7.52 (1H, dd, J=8.6, 1.9 Hz), 7.12 (1H, t, J=6.3 Hz), 6.72 (2H, s), 4.28 (2H, d, J=6.2 Hz).

### EXAMPLE 10

### N-[(4-bromobenzo[b]thien-3-yl)methyl]-sulfamide (Compound #17)

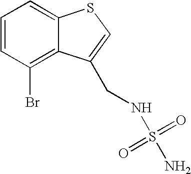

4-Bromobenzothiophene (1.80 g, 8.45 mmol) and dichloromethyl methyl ether (1.46 g, 12.7 mmol) were dissolved in anhydrous DCM (100 mL). Titanium tetrachloride (2.40 g, 12.7 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×150 mL). The extracts were concentrated and chromatographed (0 to 15% ethyl acetate in hexane) to yield 4-bromobenzothiophene-3-carboxaldehyde (0.910 g). The 4-bromobenzothiophene-3-carboxaldehyde (0.910 g, 3.77 mmol) and sulfamide (3.0 g, 31 mmol) were combined in anhydrous ethanol (25 mL) and heated to reflux for three days. The reaction was cooled to room temperature and sodium borohydride (0.157 g, 4.15 mmol) was added. After five hours, water (50 ml) was added and the solution was extracted with chloroform (3×50 mL). The extracts were concentrated, suspended in a minimal amount of DCM, and filtered to yield the title compound as a yellow solid.

¹H NMR (DMSO-d₆): δ 8.05 (1H, dd, J=8.1, 0.8 Hz), 7.78 (1H, s), 7.64 (1H, dd, J=7.6, 0.8 Hz), 7.27 (1H, t, J=7.9 Hz), 7.13 (1H, t, J=6.3 Hz), 6.72 (2H, br s), 4.65 (2H, d, J=5.3 Hz).

### EXAMPLE 11

### N-[(7-fluorobenzo[b]thien-3-yl)methyl]-sulfamide (Compound #18)

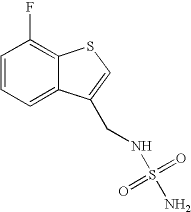

2-Fluorothiophenol (4.14 g, 32.6 mmol) was dissolved in anhydrous THF (100 mL). Potassium tert-butoxide (1.0 M in THF, 35.8 mL) was added and the suspension was stirred at room temperature for 15 minutes. 2-Chloroacetaldehyde dimethyl acetal was added and the mixture was stirred for 3 days. Water (100 mL) was added and the solution was extracted with diethyl ether (3×100 mL). The extracts were concentrated to a yellow oil and chromatographed (5 to 20% ethyl acetate in hexane) to yield 1-(2,2-dimethoxy-ethylsulfanyl)-2-fluoro-benzene (6.42 g) as a colorless oil. Chlorobenzene (25 mL) was heated to reflux and polyphosphoric acid (1 mL) was added. The 1-(2,2-dimethoxy-ethylsulfanyl)-2-fluoro-benzene was then added slowly turning the solution dark. After 3 hours of heating, the reaction was cooled to room temperature and diluted with water (50 mL). The solution was extracted with benzene (2×50 mL). The extracts were concentrated and chromatographed (0 to 15% ethyl acetate in hexane) to yield 7-fluorobenzothiophene (0.77 g). The 7-fluorobenzothiophene (0.77 g, 5.1 mmol) and dichloromethyl methyl ether (0.872 g, 7.6 mmol) were dissolved in anhydrous DCM (25 mL). Titanium tetrachloride (1.0 M in DCM, 7.6 mL, 7.6 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 7-fluorobenzothiophene-3-carboxaldehyde (0.642 g). The 7-fluorobenzothiophene-3-carboxaldehyde (0.642 g, 3.77 mmol) and sulfamide (1.7 g, 18 mmol) were combined in anhydrous ethanol (20 mL) and heated to reflux for three days. The reaction was cooled to room temperature and sodium borohydride (0.148 g, 3.92 mmol) was added. After two hours, water (25 ml) was added and the solution was extracted with chloroform (3×25 mL). The extracts were concentrated, suspended in a minimal amount of DCM, and filtered to yield the title compound as a yellow solid.

¹H NMR (DMSO-d₆): δ 7.78 (1H, d, J=8.0 Hz), 7.43-7.50 (1H, m), 7.27 (1H, dd, J=10.3, 7.9 Hz), 7.14 (1H, t, J=6.4 Hz), 6.74 (2H, brs), 4.31 (2H, d, J=6.4 Hz).

### EXAMPLE 12

### N-[(4-trifluoromethylbenzo[b]thien-3-yl)methyl]-sulfamide (Compound #19)

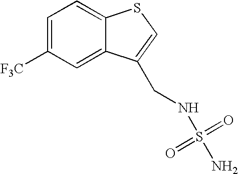

4-Trifluoromethylbenzothiophene (0.276 g, 1.37 mmol) and dichloromethyl methyl ether (0.236 g, 2.06 mmol) were dissolved in anhydrous DCM (10 mL). Titanium tetrachloride (1.0M in DCM, 2.1 mL, 2.1 mmol) was added, turning the solution dark. After 30 minutes at room temperature, the reaction was poured into a mixture of saturated aqueous NaHCO₃ and ice. The mixture was stirred for about 30 minutes and then extracted with DCM (2×25 mL). The extracts were concentrated and chromatographed (0 to 15% ethyl acetate in hexane) to yield 4-trifluoromethylbenzothiophene-3-carboxaldehyde.

The 4-trifluoromethylbenzothiophene-3-carboxaldehyde (0.226 g, 0.982 mmol) and sulfamide (0.471 g, 4.91 mmol) were combined in anhydrous ethanol (5 mL) and heated to reflux for 24 hours. The reaction was cooled to room temperature and sodium borohydride (0.056 g, 1.47 mmol) was added. After five hours, water (10 ml) was added and the solution was extracted with chloroform (3×10 mL). The extracts were concentrated, and chromatographed (5% methanol in DCM) to yield the title compound as a white solid.

¹H NMR (DMSO-d₆): δ 8.30 (1H, s), 8.25 (1H, d, J=8.4 Hz), 7.84 (1H, s), 7.68 (1H, dd, J=8.5, 1.4 Hz), 6.7-6.9 (2H, br s), 4.4-4.5 (1H, br s), 4.37 (2H, s).

### EXAMPLE 13

### N-[(4-cyanobenzo[b]thien-3-yl)methyl]-sulfamide (Compound #20)

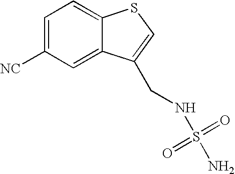

4-Cyanobenzothiophene (1.15 g, 7.22 mmol) and dichloromethyl methyl ether (1.25 g, 10.8 mmol) were dissolved in anhydrous DCM (100 mL). Titanium tetrachloride (1.0M in DCM, 10.8 mL, 10.8 mmol) was added, turning the solution dark. After 30 minutes at room temperature, the reaction was poured into a mixture of saturated aqueous NaHCO₃ and ice. The mixture was stirred for about 30 minutes and then was extracted with DCM (2×50 mL). The extracts were concentrated and chromatographed (0 to 15% ethyl acetate in hexane) to yield 4-cyanobenzothiophene-3-carboxaldehyde.

The 4-cyanobenzothiophene-3-carboxaldehyde (0.298 g, 1.59 mmol) and sulfamide (0.766 g, 7.97 mmol) were combined in anhydrous ethanol (20 mL) and heated to reflux for 24 hours. The reaction was cooled to room temperature and sodium borohydride (0.091 g, 2.39 mmol) was added. After five hours, water (20 ml) was added and the solution was extracted with chloroform (3×20 mL). The extracts were concentrated, and chromatographed (5% methanol in DCM) to yield the title compound as a white solid.

¹H NMR (DMSO-d₆): δ 8.37 (1H, s), 8.30 (1H, d, J=8.4 Hz), 7.87 (1H, s), 7.70 (1H, dd, J=8.5, 1.4 Hz), 6.7-6.9 (2H, br s), 4.4-4.5 (1H, br s), 4.40 (2H, s).

### EXAMPLE 14

### N-[(benzo[b]thien-3-yl)methyl]-sulfamoylpyrrolidine (Compound #101)

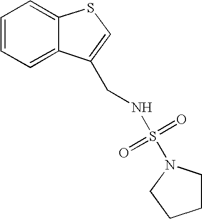

N-[(Benzo[b]thien-3-yl)methyl]-sulfamide (0.250 g, 1.03 mmol) and pyrrolidine (0.25 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.84-7.89 (2H, m), 7.38-7.45 (3H, m), 4.49 (3H, br s), 3.25 (4H, t, J=4.0 Hz), 1.80 (4H, t, J=4.0 Hz).

### EXAMPLE 15

### N-[(benzo[b]thien-3-yl)methyl]-N′-ethylsulfamide (Compound #21)

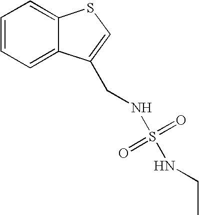

N-[(Benzo[b]thien-3-yl)methyl]-sulfamide (0.250 g, 1.03 mmol) and ethylamine (70% in H₂O, 0.10 mL) were combined in anhydrous dioxane (5 mL) and heated to reflux for 32 hours. The reaction was evaporated and chromatographed with 5% methanol in DCM to yield the title compound as a white solid.

¹H NMR (CDCl₃): δ 7.83-7.90 (2H, m), 7.36-7.47 (3H, m), 4.51 (2H, s), 2.90 (2H, q, J=7 Hz), 1.03 (3H, t, J=7 Hz).

### EXAMPLE 16

### Imidazole-1-sulfonic acid [(benzo[b]thien-3-yl)methyl]-amide (Compound #102)

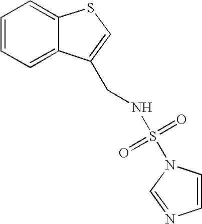

3-Benzothienylmethylamine and 3-(imidzole-1-sulfonyl)-1-methyl-3H-imidazol-1-ium triflate were combined in anhydrous acetonitrile. The solution was stirred at room temperature overnight, concentrated, and chromatographed (5% methanol in DCM) to yield the title compound as a tan solid.

¹H NMR (DMSO-d₆): δ 8.05 (1H, dd, J=7.0, 1.6 Hz), 7.99 (1H, dd, J=7.1, 1.7 Hz), 7.85 (1H, s), 7.66 (1H, s), 7.42-7.65 (5H, m), 4.34 (2H, s).

### EXAMPLE 17

### Dominant-Submissive Competition: In Vivo Assay

Dominance and submissiveness, defined in a competition test and measured as the relative success of two food-restricted rats to gain access to a feeder, form a behavioral paradigm—the Dominant Submissive Relationship (DSR). This paradigm results in two models which are predictive of ability of test compounds (drugs) to treat mood disorders.

Test compounds are evaluated for either their ability to inhibit the dominant behavior of rats taking food at the expense of an opponent (reduction of dominant behavior model or RDBM) and thus their potential as treatments for mania; or for their ability to increase the competitive behavior of submissive rats losing such encounters (reduction of submissive behavior model or RSBM) and thus their potential as treatments for depression; (Malatynska, E., and Knapp, R. J., Neuroscience and Biobehavioral Review, 29 (2005) 715-737).

The Reduction of Submissive Behavior Model (RSBM) test, wherein the submissive animals are treated with test compound, is predictive of the ability of the test compound to treat depression. This model was applied to Compound #1 of the present invention, according to the following procedure.

Male Sprague Dawley rats (140 to 160 g) from Charles River Laboratories Wilmington, Mass. were used in this assay. Shipments of rats were received at two-week intervals. Each shipment went through five-day quarantine, one-week acclimation period, and one-week selection process, followed by five-weeks of drug or vehicle treatment to those pairs selected.

Rats were housed four per cage. Access to food was restricted to one hour per day after testing on Monday through Thursday. After testing on Friday, rats had free access to food until being fasted again on Sunday. At no time were the rats deprived of water. The food deprivation periods used had little effect on weight gain as the average weight of rats was about 300 g at the end of the study. At the conclusion of experiment rats were sacrificed by decapitation, the trunk blood and brains were collected for in vitro experiments and drug concentration measurements.

The basic testing apparatus consisted of two chambers connected with a tunnel only large enough to allow one rat to pass through at a time. On the floor, at the mid-point of the tunnel was a container of sweetened milk. This basic apparatus was replicated, so that a total of four pairs of rats can be video tracked simultaneously. The camera can distinguish rats marked by different colors. Thus, the rats' heads were colored for the purpose of video tracking, red in one cage and yellow in the other cage. Only one animal at a time can have comfortable access to the feeder, but both animals can drink milk during the five-minute daily session. During the five-minute daily sessions, time spent in the feeder zone by each rat was recorded by the video tracking software and saved into a text file.

The test began with a random assignment of rats into pairs. Each member of a pair was placed in an opposite chamber of the testing apparatus. The time spent in the feeder zone by each animal was recorded. During the first week (five days) of testing the animals habituate to the new environment. Dominance was assigned to the animal with the highest score during the second week of testing if three criteria were achieved. First, there must have been a significant difference (two-tailed t-test, P<0.05) between the average daily drinking scores of both animals. Second, the dominant animal score must have been at least 25% greater than the submissive animal's score. Finally, there must have been no “reversals” during the pair selection week where the putative submissive rat out-scored its dominant partner on isolated occasions. Ideally there were minimal reversals during the acclimation week as well. About twenty-five to thirty-three percent of the initial animal pairs achieved these criteria and only these pairs were continued in the study.

Terminal blood samples (0.5-1.0 mL) were collected post experiment into heparinized tubes. Blood samples were centrifuged for cell removal, and 200 μL of plasma supernatant was then transferred to a clean vial, placed on dry ice, and subsequently stored in a −80° C. freezer prior to analysis. Two hundred microliters of acetonitrile containing internal standard was added to 100 μL of plasma or brain tissue to precipitate proteins and/or tissue residues. Samples were centrifuged and supernatant removed for analysis by liquid chromatography-triple quadruple mass spectrometry (LC-MS-MS). Calibration standards were prepared by adding appropriate volumes of stock solution directly into blank plasma or brain tissue homogenates and treated identically to collected samples. Calibration standards were prepared in the range of 0.01 to 10 μM for quantitation. LC-ESI-MS/MS (negative mode) analysis was performed utilizing multiple reaction monitoring (MRM) for detection of characteristic ions for the test compound.

Significant differences between time spent on the feeder by dominant and submissive rats were determined by ANOVA using GraphPad Prism software (GraphPad Software, Inc. San Diego, Calif.) followed by a two-tailed t-test (P<0.05). Comparisons were made between treatment groups using normalized dominance level values in paired animals. The dominance level is a value that measures social relation between paired subjects. Dominance level (DL)=FTD-FTS where FTD is the feeder time of dominant rats and FTS is the feeder time of submissive rats. The normalization was conducted according to the formula:

Dominance Level (week n in %)=(Dominance Level (week n))/(Dominance Level (week 2)

The statistical significance of the difference in dominance level between the control group (pairs of rats where both dominant and submissive animals were treated with vehicle) and the treatment group (submissive rats were treated with drug and dominant rats with vehicle) was determined by ANOVA, followed by a t-test. The activity onset time value at 50% of response (AOT-50) and the minimum and maximum response to drug were calculated based on the reduction of the dominance level value using non-linear regression analysis (GraphPad Software, Inc., San Diego, Calif.). The normalized DL values were used for this calculation, where DL values for treatment weeks were normalized as a percent of the second week (pretreatment) value of that pair according the above formula. In these settings the minimum of the response (DL) determined drug positive activity, corresponding to efficacy, since DL values were always reduced if the response to a drug was positive. In the case of the negative response to a drug (worsening of symptoms) DL values were increased. If the drug did not have such activity the maximum of the response did not exceed 100%. Any maximal DL value significantly higher then control value (about 100%) indicated drug negative activity.

Compound # 1 was evaluated in the Rat Reduction of Submissive Behavior Model (RSBM) of depression (Malatynska E, Goldenberg R, Shuck L, Haque A, Crites G, and Knapp R. Reduction of submissive behavior as a model of depression. Pharmacol. 1:1-9 2002; Malatynska, E., and Knapp, R. J., Neuroscience and Biobehavioral Review, 29 (2005) 715-737); Pinhasov, A., Crooke, J., Rosenthal, D., Brenneman D. E., and Malatynska, E. Reduction of Submissive Behavior Model for antidepressant drug activity testing: study using a video-tracking system. Behav Pharmacol. December; 16(8):657-64, 2005).

In RSBM, three groups of submissive rats were treated BID with Compound #1; one treated at 6 mg/kg (n=6), a second at 30 mg/kg (n=6) and a third at 60 mg/kg (n=7). A fourth group of submissive rats was treated with fluoxetine at 10 mg/kg/QD (n=10), and a fifth with 0.5% methylcellulose (n=6). In RDBM, four groups of dominant rats were treated BID with Compound #1; one treated at 1.25 mg/kg (n=3), a second at 6 mg/kg (n=6), a third at 30 mg/kg (n=6) and a fourth at 60 mg/kg dose (n=7). A fifth group of dominant rats was treated with lithium chloride at 100 mg/kg/QD (n=6), and a sixth with 0.5% methylcellulose (vehicle control). All treatments started on Saturday after the second testing week (selection week). Fluoxetine and lithium chloride were injected intraperitoneally (i.p.) once a day (QD). Compound #1 was administered orally (p.o) twice a day (BID). Approximate time of the first daily dose was between 7:00 a.m. and 8:00 a.m. and the second daily dose between 4:00 p.m. and 6:00 p.m.

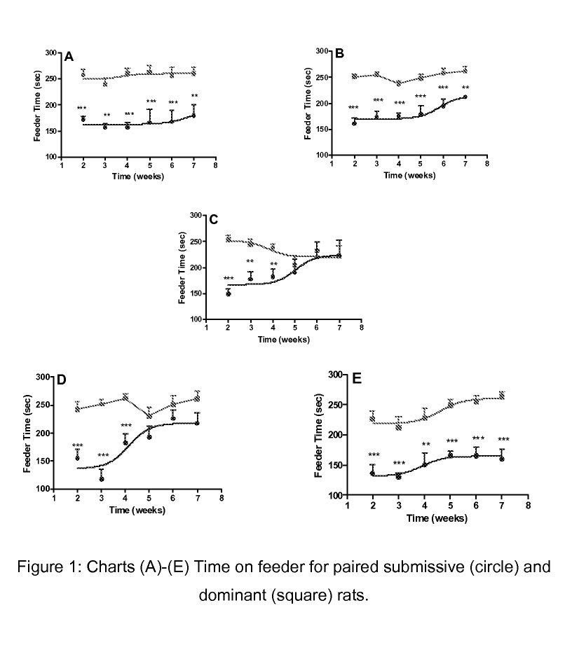

FIG. 1, in Charts (A) through (E) illustrates the time on feeder for paired submissive and dominant rats as follows: Chart (A) plots submissive rats were treated with Compound #1 at 6 mg/kg/day BID versus dominant rats treated with vehicle BID; Chart (B) plots submissive rate were treated with Compound #1 at 30 mg/kg/day BID versus dominant rats treated with vehicle BID; Chart (C) plots submissive rats were treated with Compound #1 at 60 mg/kg/day BID versus dominant rats treated with vehicle BID. For comparison Chart (D) plots submissive rats were treated with fluoxetine at 10 mg/kg/day QD versus dominant rats treated with water QD; and Chart (E) plots submissive and dominant rats treated with vehicle. In all the charts, submissive animals are plotted as circle, dominant rats are plotted as square. In the charts, the designation “**” indicated a statistical difference with p<0.01 and a designation of “***” indicates a statistical difference with p<0.001.

Compound #1 reduced submissive behavior in a dose-dependent manner indicating that the compound is active as an antidepressant. The reduction of submissive behavior was statistically significant after 17 days of treatment with 60 mg/kg (p<0.01). Submissive rats treated with increasing doses of Compound #1 further displayed a linear increase in exposure both in plasma and brain.

### EXAMPLE 18

### Mouse Tail Suspension In Vivo Assay

The tail suspension test (TST) is an acute test of anti-depressant activity.

Male NMRI mice (25-30 g; n=13-15 mice per dose) were given a single dose of vehicle (aqueous solution of 1 equivalent tartaric acid+0.45% NaCl+10% cyclodextrin, i.p.), imipramine (20 mg/kg, i.p. in an aqueous solution of 0.9% NaCl) or Compound #1 (at 5, 10, 20, 40 or 80 mg/kg, i.p.) 30 min prior to tail suspension. In this test, the mice were submitted to an unpleasant and inescapable situation (hanging by the tail) for 6 min. Once suspended, motor activity diminished rapidly and the mice became immobile. A compound was considered active as an antidepressant in this model, when there was a reduction in immobility time. Viewpoint video-tracking software was utilized to record immobility time.

It is important to note that the NMRI mice used in this test, do not respond to all antidepressants in this model; rather they exhibit selective sensitivity to 5-HT reuptake inhibitors and some tricyclics. Therefore, a compound which is not active in this model may nonetheless be active as an antidepressant. Inactivity in this model would only suggest that the compound does not inhibit 5-HT reuptake.

Compound #1 did not reduce immobility time up to 40 mg/kg (vehicle=85 sec; Compound #1=66 to 101 sec; p=1.0). At 80 mg/kg, a significant increase in immobility time was observed which resembled sedation (vehicle=85 sec; 80 mg/kg Compound #1=160 sec; p=0.003). Imipramine, a tricyclic antidepressant, caused a significant decrease (75%) in immobility time (vehicle=71 sec; 20 mg/kg imipramine=18 sec; p=0.0004). Results from this assay are as listed in Table 3 below.

TABLE 3
Immobility Time in the Mouse Tail Suspension Test

| | | | | Immobilization Time |
|---|---|---|---|---|
| Compound | Dose (mpk) | Route | # Animals | (sec); (mean ± SEM) |
| #1 | 0 | i.p. | 14 | 84.9 ± 14.1 |
| #1 | 5 | i.p. | 15 | 66.1 ± 10.1 |
| #1 | 10 | i.p. | 14 | 96.4 ± 12.4 |
| #1 | 20 | i.p. | 15 | 110.1 ± 15.8 |
| #1 | 40 | i.p. | 15 | 100.5 ± 13.9 |
| #1 | 80 | i.p. | 14 | 160.4 ± 10.7 |
| Imipramine | 0 | i.p. | 13 | 71.0 ± 15.5 |
| Imipramine | 20 | i.p. | 14 | 18.0 ± 5.8 |

### EXAMPLE 19

### Mouse Forced Swim Test

The forced swim test (FST) is an acute test of anti-depressant activity. Note that in this test, antidepressant-like compounds such as tricyclics, MAO inhibitors, SSRIs and others show activity, although the level of measured activity can vary with strain differences.

Male C57BL/6 mice (˜25 g; n=10 mice per dose) were given a single dose of vehicle (an aqueous solution of 1 equivalent tartaric acid+0.45% NaCl+10% cyclodextrin, s.c.), Compound #1 (at 5, 10, 20, 40 or 80 mg/kg, s.c.) or imipramine (10 mg/kg, i.p. in an aqueous solution of 0.9% NaCl) 30 min prior to entrance into the swim chamber. In this test, mice were placed in a water-filled cylinder in which they were unable to escape or touch the bottom of the chamber for 6 min. Once immersed in water, motor activity diminished rapidly and the mice became immobile. Viewpoint video-tracking software was utilized to record immobility time. Other common behaviors such as swimming and climbing activity were not measured in this study.

Treatment with Compound #1 resulted in increased immobility time at all doses (vehicle=152 sec; Compound #1=179 to 193 sec) that was not statistically significant (p=0.26 to 1.0). Imipramine caused a modest decrease (20%) in immobility time (vehicle=152 sec; 10 mg/kg imipramine=122 sec) that was similarly not statistically significant (p=0.19).

### EXAMPLE 20

As a specific embodiment of an oral composition, 100 mg of the Compound #1 prepared as in Example 1 is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A method of treating depression comprising administering to a subject in need thereof a therapeutically effective amount of a compound of the formula (I) 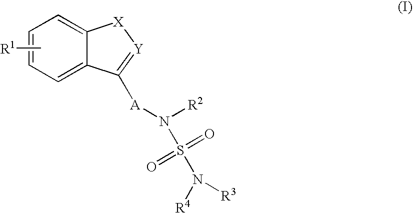
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

2. The method of claim 1 wherein
R¹ is selected from the group consisting of hydrogen, halogen, trifluoromethyl, cyano and nitro;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen, methyl and ethyl;
or a pharmaceutically acceptable salt thereof.

3. The method of claim 2, wherein
R¹ is selected from the group consisting of hydrogen, halogen, trifluoromethyl and cyano;
X—Y is selected from the group consisting of —S—CH—, —O—CH—, —O—C(CH₃)—, , —N(CH₃)—CH— and —CH═CH—CH—;
A is selected from the group consisting of —CH₂— and —CH(CH₃)—;
R² is hydrogen;
R³ and R⁴ are each independently selected from the group consisting of hydrogen and ethyl;
or a pharmaceutically acceptable salt thereof.

4. The method of claim 3, wherein
R¹ is selected from the group consisting of hydrogen, 5-chloro, 5-fluoro, 5-bromo, 4-bromo, 7-fluoro, 5-trifluoromethyl and 5-cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is hydrogen;
R³ and R⁴ are each hydrogen; alternatively R³ is hydrogen and R⁴ is ethyl;
or a pharmaceutically acceptable salt thereof.

5. The method of claim 1, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

6. The method of claim 5, wherein
R¹ is selected from the group consisting of hydrogen, halogen, trifluoromethyl and cyano;
X—Y is selected from the group consisting of —S—CH—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are taken together with the nitrogen atom to which they are bound to form a 5 to 6 membered, saturated or aromatic ring structure, optionally containing one to two additional heteroatoms independently selected from the group consisting of O, N and S;
or a pharmaceutically acceptable salt thereof.

7. The method of claim 6, wherein
R¹ is hydrogen;
X—Y is —S—CH—;
A is —CH₂—;
R² is hydrogen;
R³ and R⁴ are taken together with the nitrogen atom to which they are bound to form a 5 membered ring structure selected from the group consisting of pyrrolidinyl and imidazolyl;
or a pharmaceutically acceptable salt thereof.

8. The method of claim 2, wherein the compound of formula (I) is selected from the group consisting of
N-(benzo[b]thien-3-yl methyl)-sulfamide;
N-[(5-chlorobenzo[b]thien-3-yl)methyl]-sulfamide;
N-(3-benzofuranylmethyl)-sulfamide;
N-[(5-fluorobenzo[b]thien-3-yl)methyl]-sulfamide;
N-(1-benzo[b]thien-3-ylethyl)-sulfamide;
N-(1-naphthalenylmethyl)-sulfamide;
N-[(2-methyl-3-benzofuranyl)methyl]-sulfamide;
N-[(5-bromobenzo[b]thien-3-yl)methyl]-sulfamide;
N-[(4-bromobenzo[b]thien-3-yl)methyl]-sulfamide;
N-[(7-fluorobenzo[b]thien-3-yl)methyl]-sulfamide;
N-[(1-methyl-1H-indol-3-yl)methyl]-sulfamide;
N-[(4-trifluoromethylbenzo[b]thien-3-yl)methyl]-sulfamide;
N-[(4-cyanobenzo[b]thien-3-yl)methyl]-sulfamide;
N-[(benzo[b]thien-3-yl)methyl]-sulfamoylpyrrolidine;
N-[(benzo[b]thien-3-yl)methyl]-N′-ethylsulfamide;
imidazole-1-sulfonic acid [(benzo[b]thien-3-yl)methyl]-amide;
and pharmaceutically acceptable salts thereof.

9. The method of claim 1, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

10. A method of treating depression comprising administering to a subject in need thereof a therapeutically effective amount of a compound selected from the group consisting of N-(benzo[b]thien-3-ylmethyl)-sulfamide and pharmaceutically acceptable salts thereof.

11. The method of claim 1, wherein the depression is selected from the group consisting of major depressive disorder, unipolar depression, treatment refractory depression, resistant depression, anxious depression and dysthymia.

12. The method of claim 1, wherein the depression is selected from the group consisting of major depressive disorder, unipolar depression, treatment refractory depression, resistant depression and anxious depression.

13. The method of claim 1, wherein the depression is selected from the group consisting of major depressive disorder, unipolar depression, treatment refractory depression, resistant depression and anxious depression.

14. The method of claim 1, wherein the depression is major depressive disorder.

15. The method of claim 10, wherein the depression is selected from the group consisting of major depressive disorder, unipolar depression, treatment refractory depression, resistant depression, anxious depression and dysthymia.

16. The method of claim 10, wherein the depression is selected from the group consisting of major depressive disorder, unipolar depression, treatment refractory depression, resistant depression and anxious depression.

17. The method of claim 10, wherein the depression is selected from the group consisting of major depressive disorder, unipolar depression, treatment refractory depression, resistant depression and anxious depression.

18. The method of claim 10, wherein the depression is major depressive disorder.

19. A method for the treatment of depression comprising administering to a subject in need thereof co-therapy with a therapeutically effective amount of at least one antidepressant and a compound of formula (I) 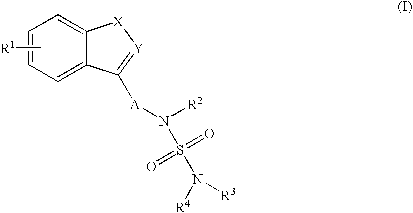
wherein
R¹ is selected from the group consisting of hydrogen, halogen, hydroxy, methoxy, trifluoromethyl, nitro and cyano;
X—Y is selected from the group consisting of —S—CH—, —S—C(CH₃)—, —O—CH—, —O—C(CH₃)—, —N(CH₃)—CH— and —CH═CH—CH—;
A is selected from the group consisting of —CH₂— and —CH(CH₃)—;
R² is selected from the group consisting of hydrogen and methyl;
R³ and R⁴ are each independently selected from the group consisting of hydrogen and C₁₋₄alkyl;
alternatively, R³ and R⁴ are taken together with the nitrogen atom to which they are bound to form a 5 to 7 membered, saturated, partially unsaturated or aromatic ring structure, optionally containing one to two additional heteroatoms independently selected from the group consisting of O, N and S;
or a pharmaceutically acceptable salt thereof.

20. The method of claim 19, wherein the compound of formula (I) is selected from the group consisting of N-(benzo[b]thien-3-ylmethyl)-sulfamide; N-[(5-fluorobenzo[b]thien-3-yl)methyl]-sulfamide; and pharmaceutically acceptable salts thereof.

21. The method of claim 19, wherein the antidepressant is selected from the group consisting of imipramine, amitriptyline, desipramine, nortriptyline, doxepin, protriptyline, trimipramine, maprotiline, amoxapine, trazodone, bupropion, chlomipramine, fluoxetine, duloxetine, escitalopram, citalopram, sertraline, paroxetine, fluvoxamine, nefazadone, venlafaxine, milnacipran, reboxetine, mirtazapine, pheneizine, tranylcypromine, moclobemide, Kava-Kava, St. John's Wart, s-adenosylmethionine, thyrotropin releasing hormone, neurokinin receptor antagonists and triiodothyronine.

22. The method of claim 19, wherein the antidepressant is selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

23. The method of claim 19, wherein the antidepressant is selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

24. The method of claim 19, wherein the antidepressant is selected from the group consisting of neuropeptides, compounds targeting neuropeptide receptors and hormones.

25. A method for the treatment of depression comprising administering to a subject in need thereof co-therapy with a therapeutically effective amount of at least one antidepressant and a compound selected from the group consisting N-(benzo[b]thien-3-ylmethyl)-sulfamide; and pharmaceutically acceptable salts thereof.

26. The method of claim 25, wherein the antidepressant is selected from the group consisting of imipramine, amitriptyline, desipramine, nortriptyline, doxepin, protriptyline, trimipramine, maprotiline, amoxapine, trazodone, bupropion, chlomipramine, fluoxetine, duloxetine, escitalopram, citalopram, sertraline, paroxetine, fluvoxamine, nefazadone, venlafaxine, milnacipran, reboxetine, mirtazapine, phenelzine, tranylcypromine, moclobemide, Kava-Kava, St. John's Wart, s-adenosylmethionine, thyrotropin releasing hormone, neurokinin receptor antagonists and triiodothyronine.

27. The method of claim 25, wherein the antidepressant is selected from the group consisting of mono-amine oxidase inhibitors, tricyclics, serotonin reuptake inhibitors, serotonin noradrenergic reuptake inhibitors; noradrenergic and specific serotonergic agents and atypical antidepressants.

28. The method of claim 25, wherein the antidepressant is selected from the group consisting of phenelzine, tranylcypromine, moclobemide, imipramine, amitriptyline, desipramine, nortriptyline, doxepin, protriptyline, trimipramine, chlomipramine, amoxapine, fluoxetine, sertraline, paroxetine, citalopram, fluvoxamine, venlafaxine, milnacipran, mirtazapine and bupropion.

29. The method of claim 25, wherein the antidepressant is selected from the group consisting of neuropeptides, compounds targeting neuropeptide receptors and hormones.
